(19) United States
(12) Patent Application Publication
(10) Pub. No.: US 2008/0118586 A1
(43) Pub. Date: May 22, 2008

(54) Medicated cream for topical application

(76) Inventor: Esterina Difrank, Pittsburgh, PA (US)

Correspondence Address: JAMES RAY & ASSOCIATES, 2640 PITCAIRN ROAD, MONROEVILLE, PA, 15146, US

(21) Appl. No.: 11/602,415

(22) Filed: Nov. 20, 2006

(51) Int. Cl. A61K 36/886; A61K 31/58; A61P 17/02

(52) U.S. Cl. 424/744; 514/171

(57) ABSTRACT

A topical cream specially formulated for the treatment of at least one of insect bites, skin irritations and rashes. Such topical cream comprising corticosteroid cream present therein in an amount of between about 40.0 weight percent to about 60.0 weight percent, a moisturizing cream present therein in an amount of between about 20.0 weight percent to about 30.0 weight percent and an aloe vera gel present therein in an amount of between about 20.0 weight percent to about 30.0 weight percent.

### FIELD OF THE INVENTION

The present invention relates, in general, to medicated type topical creams and lotions for soothing irritated skin and, more particularly, this invention relates to a medicated type cream formulation that has been specifically formulated for at least one of alleviating the itching, inflammation and/or burning caused by insect bites, skin irritations and various rashes thus aiding in the healing process when such cream is applied to the affected area.

### BACKGROUND OF THE INVENTION

Prior to the conception and development of the present invention, as is generally well recognized in the prior art, various types of ointments and lotions having some medications mixed therein have been sold over the counter, as well as by prescription, to help relieve the itching and/or burning sensation caused by certain insects bites, diaper rash, poison ivy and certain allergies.

Obviously, as would generally be expected, some of these prior art type ointments and lotions are more effective than others in treating the specific symptoms being exhibited by a person having these different undesirable afflictions. For example only, calamine lotion is widely utilized to treat poison ivy and baby lotion is widely used to treat diaper rash on infants. However, calamine lotion is not at all effective in treating diaper rash, nor is baby lotion effective at all in the treatment of poison ivy. Furthermore, calamine lotion has a pink color which may not be desirable if it cannot be covered up with clothing and the person must go out in public. This is especially the case with people who are self conscious about there appearance.

Because there is no one particular ointment or lotion presently available suitable for treating the many different afflictions there are numerous such items currently being sold many of which are totally ineffective for the problem at hand.

### SUMMARY OF THE INVENTION

The present invention, in a first aspect, provides a topical cream specially formulated for the treatment of at least one of insect bites, skin irritations and rashes. Such topical cream comprising a corticosteroid cream present in said topical cream in an amount of between about 40.0 weight percent to about 60.0 weight percent. There is also a moisturizing cream present in such topical cream in an amount of between about 20.0 weight percent to about 30.0 weight percent and an aloe vera gel is present in the topical cream in an amount of between about 20.0 weight percent to about 30.0 weight percent.

The present invention, in a second aspect, provides a method of making a topical cream specially formulated for the treatment of at least one of insect bites, skin irritations and rashes. The method includes the steps of placing an amount of corticosteroid cream into a mixer wherein such amount is between about 40.0 weight percent to about 60.0 weight percent of the final formulation amount. Placing an amount of a moisturizing cream into such mixer with such amount being between about 20.0 weight percent to about 30.0 weight percent of the final formulation amount. Placing an amount of an aloe vera gel into the mixer wherein such amount is between about 20.0 weight percent to about 30.0 weight percent of the final formulation and thereafter mixing such corticosteroid cream, such moisturizing cream and such aloe vera gel together to produce such topical cream specially formulated for the treatment of at least one of insect bites, skin irritations and rashes.

In a third and final aspect of the present invention there is taught a method of treating at least one of insect bites, skin irritations and rashes. The method comprises the step of applying a thin film of a topical cream having corticosteroid cream present therein in an amount of between about 40.0 weight percent to about 60.0 weight percent, a moisturizing cream present therein in an amount of between about 20.0 weight percent to about 30.0 weight percent and an aloe vera gel present therein in an amount of between about 20.0 weight percent to about 30.0 weight percent two to three times a day.

### OBJECTS OF THE INVENTION

It is, therefore, one of the primary objects of the present invention to provide a cream specifically formulated to relieve the itching, inflammation and burning caused by certain insect bites, skin irritations and various type rashes.

An additional object of the present invention is to provide a cream specifically formulated to relieve the itching, inflammation and burning which can be applied topically to the affected area of the body.

Still another object of the present invention is to provide a cream that is specifically formulated to relieve the itching, inflammation and burning and which is more effective against a larger number of afflictions than are most prior art type ointments and lotions.

Yet another object of the present invention is to provide a cream specifically formulated to relieve the itching, inflammation and burning which exhibits quicker acting traits than the prior art type ointments and lotions.

A still further object of the present invention is to provide a cream specifically formulated to relieve the itching, inflammation and burning which has a pleasant odor.

An additional object of the present invention is to provide a cream specifically formulated to relieve the itching, inflammation and burning which is easy to manufacture.

In addition to the various objects and advantages of the present invention described with some degree of specificity above it should be obvious that additional objects and advantages of the present invention will become more readily apparent to those persons who are skilled in the relevant art from the following more detailed description of the invention, particularly, when such description is taken in conjunction with the attached drawing figures and with the appended claims.

### BRIEF DESCRIPTION OF A PRESENTLY PREFERRED AND VARIOUS ALTERNATIVE EMBODIMENTS OF THE INVENTION

The present invention provides a topical cream that is specially formulated for the treatment and/or relief of itching, inflammation and discomfort caused by at least one of insect bites, skin irritations and rashes. This topical cream includes a corticosteroid cream present in such topical cream formulation in an amount of between about 40.0 weight percent to about 60.0 weight percent. In the presently preferred embodiment of the invention such corticosteroid cream is a triamcinolone acetonide 0.1% cream. If the corticosteroid cream is present in an amount of less than about 40.0 weight percent the formulation will be much less effective and if the corticosteroid cream is present in an amount greater than about 60.0 weight percent the formulation becomes more expensive.

In the broadest sense, this corticosteroid cream is present in such topical cream formulation in an amount of generally between about 45.0 weight percent to about 55.0 weight percent. It is most preferred that such corticosteroid cream will be present in such topical cream formulation in an amount of between about 45.0 weight percent to about 50.0 weight percent.

The topical cream further includes a moisturizing cream which is present in such topical cream formulation in an amount of between about 20.0 weight percent to about 30.0 weight percent. The most preferred moisturizing cream for use in the formulation for the topical cream specially formulated for the treatment and/or relief of itching, inflammation and discomfort caused by at least one of insect bites, skin irritations and rashes is Vaseline of the deep moisture creamy formula. It is also presently preferred for such moisturizing cream to include vitamin E.

Envisioned in a somewhat narrower range such moisturizing cream will be present in such topical cream formulation generally in an amount of between about 22.5 weight percent to about 27.5 weight percent. In the presently most preferred embodiment of the invention, such moisturizing cream will be normally be present in such topical cream formulation in an amount of between about 22.5 weight percent to about 25.0 weight percent.

The final essential element of the topical cream specially formulated for the treatment and/or relief of itching, inflammation and discomfort caused by at least one of insect bites, skin irritations and rashes is an aloe vera gel present in such topical cream in an amount of generally between about 20.0 weight percent to about 30.0 weight percent.

In a more preferred embodiment of the invention such aloe vera gel will be present in such topical cream formulation generally in an amount of between about 22.5 weight percent to about 27.5 weight percent. It is most preferred that such aloe vera gel to be present in the topical cream formulation in an amount of between about 22.5 weight percent to about 25.0 weight percent. It is also within the scope of the present invention for the topical cream to further include vitamin E.

The present invention further provides a method of making a topical cream that is specially formulated for the treatment of at least one of insect bites, skin irritations and rashes.

This method includes the steps of placing a predetermined amount of corticosteroid cream into a mixer. such predetermined amount generally being between about 40.0 weight percent to about 60.0 weight percent of the final formulation amount.

Further, according to the method a predetermined amount of a moisturizing cream is placed into such mixer. Such predetermined amount of the moisturizing cream generally being between about 20.0 weight percent to about 30.0 weight percent of the final formulation amount. Additionally, there is a predetermined amount of an aloe vera gel placed into such mixer. The predetermined amount of aloe vera gel is generally between about 20.0 weight percent to about 30.0 weight percent of the final formulation.

The final essential step, according to the method, is mixing the corticosteroid cream and the moisturizing cream and such aloe vera gel together to produce such topical cream specially formulated for the treatment of at least one of insect bites, skin irritations and rashes.

In the presently preferred method, prior to mixing there is added a predetermined amount of vitamin E to such mixer. It is within the scope of the present invention for the moisturizing cream to include such vitamin E. The presently preferred corticosteroid cream is triamcinolone acetonide 0.1% cream.

Last, the present invention teaches a method of treating at least one of insect bites, skin irritations and rashes. According to this method there is applied thin film of a topical cream having corticosteroid cream present therein in an amount of between about 40.0 weight percent to about 60.0 weight percent, a moisturizing cream present therein in an amount of between about 20.0 weight percent to about 30.0 weight percent and an aloe vera gel present therein in an amount of between about 20.0 weight percent to about 30.0 weight percent two to three times a day.

While a presently preferred and various alternative embodiments of the present invention have been described in sufficient detail above to enable a person skilled in the relevant art to make and use the same it should be obvious that various other adaptations and modifications can be envisioned by those persons skilled in such art without departing from either the spirit of the invention or the scope of the appended claims.

I claim:

1. A topical cream specially formulated for the treatment of at least one of insect bites, skin irritations and rashes, said topical cream comprising:
(a) a corticosteroid cream present in said topical cream in an amount of between about 40.0 weight percent to about 60.0 weight percent;
(b) a moisturizing cream is present in said topical cream in an amount of between about 20.0 weight percent to about 30.0 weight percent; and
(c) an aloe vera gel is present in said topical cream in an amount of between about 20.0 weight percent to about 30.0 weight percent.

2. A topical cream, according to claim 1, wherein said corticosteroid cream is triamcinolone acetonide 0.1% cream.

3. A topical cream, according to claim 1, wherein said corticosteroid cream is present in said topical cream in an amount of between about 45.0 weight percent to about 55.0 weight percent.

4. A topical cream, according to claim 3, wherein said corticosteroid cream is present in said topical cream in an amount of between about 45.0 weight percent to about 50.0 weight percent.

5. A topical cream, according to claim 1, wherein said moisturizing cream is Vaseline of the deep moisture creamy formula.

6. A topical cream, according to claim 1, wherein said moisturizing cream includes vitamin E.

7. A topical cream, according to claim 1, wherein said moisturizing cream is present in said topical cream in an amount of between about 22.5 weight percent to about 27.5 weight percent.

8. A topical cream, according to claim 7, wherein said moisturizing cream is present in said topical cream in an amount of between about 22.5 weight percent to about 25.0 weight percent.

9. A topical cream, according to claim 1, wherein said aloe vera gel is present in said topical cream in an amount of between about 22.5 weight percent to about 27.5 weight percent.

10. A topical cream, according to claim 9, wherein said aloe vera gel is present in said topical cream in an amount of between about 22.5 weight percent to about 25.0 weight percent.

11. A topical cream, according to claim 11, wherein said topical cream further includes vitamin E.

12. A method of making a topical cream specially formulated for the treatment of at least one of insect bites, skin irritations and rashes, said method comprising the steps of:
(a) placing an amount of corticosteroid cream into a mixer, said amount being between about 40.0 weight percent to about 60.0 weight percent of the final formulation amount;
(b) placing an amount of a moisturizing cream into said mixer, said amount being between about 20.0 weight percent to about 30.0 weight percent of the final formulation amount;
(c) placing an amount of an aloe vera gel into said mixer, said amount being between about 20.0 weight percent to about 30.0 weight percent of the final formulation; and
(d) mixing said corticosteroid cream and moisturizing cream and said aloe vera gel together to produce said topical cream specially formulated for the treatment of at least one of insect bites, skin irritations and rashes.

13. The method, according to claim 12, wherein said method includes the additional step of adding a predetermined amount of vitamin E to said mixer prior to step (d).

14. The method, according to claim 13, wherein said moisturizing cream includes said vitamin E.

15. The method, according to claim 12, wherein said corticosteroid cream is triamcinolone acetonide 0.1% cream.

16. A method of treating at least one of insect bites, skin irritations and rashes, said method comprising the step of applying a thin film of a topical cream having corticosteroid cream present therein in an amount of between about 40.0 weight percent to about 60.0 weight percent, a moisturizing cream present therein in an amount of between about 20.0 weight percent to about 30.0 weight percent and an aloe vera gel present therein in an amount of between about 20.0 weight percent to about 30.0 weight percent two to three times a day.
